(19) United States
(12) Patent Application Publication
(10) Pub. No.: US 2023/0174548 A1
(43) Pub. Date: Jun. 8, 2023

(54) SYMBIOTIC PRODRUGS FOR THE TREATMENT OF CANCER AND OTHER DISEASES

(71) Applicant: BOARD OF REGENTS OF THE UNIVERSITY OF NEBRASKA, Lincoln, NE (US)

(72) Inventor: Amarnath Natarajan, Elkhorn, NE (US)

(21) Appl. No.: 17/912,706

(22) PCT Filed: Mar. 19, 2021
(86) PCT No.: PCT/US21/23077

§ 371 Date: Sep. 19, 2022

Related U.S. Application Data
(60) Provisional application No. 62/992,011, filed on Mar. 19, 2020.

(51) Int. Cl. C07D 491/107; C07D 519/00; C07D 493/04; A61P 35/00; A61P 15/08

(52) U.S. Cl. CPC C07D 491/107; C07D 519/00; C07D 493/04; A61P 35/00; A61P 15/08

(57) ABSTRACT

Provided herein are compounds and methods for modulating the NFKB pathway. More particularly, provided are inhibitors of the NFkB pathway and the uses of such inhibitors in regulating diseases and disorders, e.g., to treat cancer, such as ovarian cancer.

### STATEMENT OF U.S. GOVERNMENT SUPPORT

This invention was made with government support under grant number R01CA197999 awarded by the National Institutes of Health. The government has certain rights in the invention.

### BACKGROUND

Prodrug approaches to overcome high toxicity among chemotherapeutic agents have yielded promising results in preclinical and clinical settings. Widely used drugs such as doxorubicin are effective anticancer agents but suffer from offsite effects such as cardiomyopathy. Aldoxorubicin (INNO-206) is a prodrug of doxorubicin that exploits the acidic tumor microenvironment to trigger the release of doxorubicin. The cardiotoxicity profile of INNO-206 was far superior to doxorubicin in Phase II and III clinical trials.

Another class of anticancer agents are pathway specific inhibitors that possess enone moieties which target the sulfhydryl groups of surface exposed cysteine residues. In particular sesquiterpene natural products that contain an α-methylene-γ-butyrolactone moiety that are known to form covalent adducts with proteins in the NFκB pathway. Parthenolide and isohelenin are early sesquiterpene lactones that were identified as NFκB pathway inhibitors. In an oral administered phase I trial, parthenolide was not detected in the plasma suggesting stability issues (Curry et al., 2004). Secondary amine-based prodrugs that masked the reactive enone of parthenolide improved stability while maintaining anticancer activity in tumor models (Hexum et al., 2015; Ren, Yu, & Kinghorn, 2016).

The antibacterial field was the first to design “mutual prodrugs” also known as “codrugs” wherein two drugs are conjugated through a labile linker. The marketed codrug sultamicillin, upon hydrolysis, releases ampicillin (β-lactamase antibiotic) and penicillanic acid sulfone (β-lactamase inhibitor). Bacteria develop resistance to β-lactamase antibiotics by elevating the expression of β-lactamase, which is targeted by penicillanic acid sulfone in sultamicillin thus restoring the efficacy of ampicillin.

Prodrugs inherently have far less to no pharmacological activity when compared to the active drug but possess structural motifs that are liable to bioconversion to reveal the active drug. The release of the active drug from the prodrug also generates a pro-fragment that typically has no biological activity and, in embodiments, could result in adverse effects. Accordingly, there is a need for improved mutual prodrugs comprising one or more drugs, wherein each drug acts as a pro-fragment of the other drug, thereby eliminating the pro-fragment in the prodrug.

Inhibition of the nuclear translocation of NFκB is hypothesized to be a viable therapeutic approach for cancer, and as such, the development of drugs providing novel approaches to delivering NFκB inhibitors is becoming an important strategy in addressing the need for additional therapies for the treatment of these disorders.

### SUMMARY

The disclosure provides compounds of Formula I and pharmaceutically acceptable salts thereof:

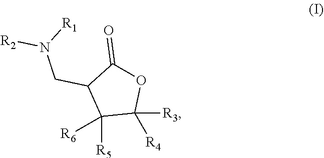

wherein each of R₁ and R₂ is independently C₁₋₆ alkyl or C₁₋₆ alkylene-Y₁—X—Y₂—Z, or R₁ and R₂ together with the nitrogen atom to which they are attached form a 5-7 membered heterocycloalkyl ring optionally having 1 additional ring heteroatom selected from O, S, and N, wherein the heterocycloalkyl ring is substituted with —CF₃ or —Y₁—X—Y₂—Z; R₃ is H; or R₃ and R₄ together with the carbon atom to which they are attached form a 5-7 membered heterocycloalkyl ring having 1-2 ring heteroatoms selected from O, S, and N, wherein the heterocycloalkyl ring is optionally fused to a C₆₋₁₀ aryl ring; R₅ is H, or R₅ and R₄ together with the carbon atoms to which they are attached form a C₆₋₁₂ cycloalkyl, wherein the cycloalkyl ring is optionally fused to a 3-5 membered heterocycloalkyl ring having 1-2 ring heteroatoms selected from O, S, and N, and the cycloalkyl ring is optionally substituted with 1-2 R₇; wherein one of R₃ and R₅ is not H; R₆ is H; each R₇ is independently H or C₁₋₆ alkyl; each of Y₁ and Y₂ is independently a bond, —NR₇—, or —C(O)NR₇—; X is 5-6 membered heteroaryl having 1-2 ring heteroatoms selected from O, S, and N, or C₆₋₁₀ aryl; Z is 6-10 membered heteroaryl having 1-3 ring heteroatoms selected from O, S, and N, 12-14 membered heterocycloalkyl having 1-3 ring heteroatoms selected from O, S, and N, or C₆₋₁₀ aryl, each substituted with 1-4 R₃; and each R₃ is independently halo, OH, C₁₋₆ alkyl, C₅₋₆ cycloalkyl, C(O)NH₂, or C(O)CH₃. In embodiments, each of R₁ and R₂ is independently C₁₋₆ alkyl, or R₁ and R₂ together with the nitrogen atom to which they are attached form a 5-7 membered heterocycloalkyl ring optionally having 1 additional ring heteroatom selected from O, S, and N, wherein the heterocycloalkyl ring is substituted with —CF₃ or —Y₁—X—Y₂—Z; R₃ is H; or R₃ and R₄ together with the carbon atom to which they are attached form a 5-7 membered heterocycloalkyl ring having 1-2 ring heteroatoms selected from O, S, and N, wherein the heterocycloalkyl ring is optionally fused to a C₆₋₁₀ aryl ring; R₅ is H, or R₅ and R₄ together with the carbon atoms to which they are attached form a C₆₋₁₂ cycloalkyl, wherein the cycloalkyl ring is optionally fused to a 3-5 membered heterocycloalkyl ring having 1-2 ring heteroatoms selected from O, S, and N, and the cycloalkyl ring is optionally substituted with 1-2 R₇; wherein one of R₃ and R₅ is not H; R₆ is H; each R₇ is independently H or C₁₋₆ alkyl; each of Y₁ and Y₂ is independently a bond, —NR₇—, or —C(O)NR₇—; X is 5-6 membered heteroaryl having 1-2 ring heteroatoms selected from O, S, and N; Z is 6-10 membered heteroaryl having 1-3 ring heteroatoms selected from O, S, and N, or C₆₋₁₀ aryl, each substituted with 1-4 R₈; and each R₈ is independently halo, OH, C₁₋₆ alkyl, C₅₋₆ cycloalkyl, or C(O)CH₃.

Other aspects of the disclosure include a compound as disclosed herein for use in the preparation of a medicament for treating or preventing a disease or disorder in a subject, and the use of a compound as disclosed herein in a method of treating or preventing a disease or disorder in a subject.

### BRIEF DESCRIPTION OF THE DRAWINGS

FIGS. 1A-1C show the stability and kinetics of release studies with a prodrug (compound 19-AP1) of the NFκB inhibitor (compound 19). FIG. 1A shows structures of the NFκB inhibitor compound 19 and its amino prodrug compounds 19-AP1 and 19AP2. FIG. 1B shows a cysteine reactivity assay used to compare the relative reactivity of compounds 19-AP1 and 19 towards cysteine. FIG. 1C shows a ¹⁹F NMR study to monitor the kinetics of the conversion of compound 19-AP1 to compound 19 as a function of free 4-trifluoromethylpiperidine.

FIGS. 2A and 2B show the synthesis of an alkyne-tagged analog of 19-AP1 (compound 4) and cellular target engagement studies. FIG. 1A shows a synthetic scheme that was used for the generation of the alkyne-tagged 19-AP1 analog (compound 4). FIG. 1B shows a flow chart for the treatment of ovarian cancer cells (A2780) with compound 4.

FIGS. 3A-3E show the results of screening assays for growth inhibitory activity. Summary of growth inhibition assays in FTE282E1 (3A), OVSAHO (3B), HPNE (3C), MiaPaCa2 (3D) and S2013 (3E) cell lines with palbociclib, AT7519, compound 19, parthenolide, (1:1) combinations and the corresponding compounds of the disclosure (compounds 5-8) (n=3, bar graph is Mean±SD). Data represents six concentrations per combination (left to right: 20, 10, 5, 2.5, 1.25, and 0.625 μM).

### DETAILED DESCRIPTION

Provided herein are novel small molecule inhibitors of NFκB and cyclin dependent kinases (CDKs). Inhibition of both the NFκB and CDK pathways have shown promise in the treatment of cancers. The compounds of the present disclosure can include small molecule prodrugs combining an NFκB inhibitor and a CDK inhibitor, allowing for the use of one composition to simultaneously inhibit two separate targets for the treatment of a disease. When administered to a patient, the prodrug can be cleaved to separate the NFκB inhibitor and the CDK inhibitor. Cleavage of the prodrug composition into the separate NFκB and CDK inhibitors can be immediate, or can take place over time, allowing for a slow release of the two separate inhibitors.

Embodiments of the compounds disclosed herein can be useful in the treatment of a variety of diseases and disorders, including but not limited to cancer, autoimmune diseases, inflammatory diseases, diabetes, cardiovascular diseases, or neurological diseases.

### Compounds of the Disclosure

In embodiments, the compound is of Formula I:

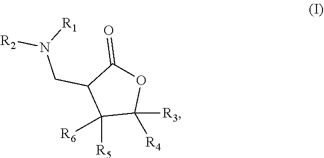

wherein

each of R₁ and R₂ is independently C₁₋₆ alkyl or C₁₋₆ alkylene-Y₁—X—Y₂—Z, or R₁ and R₂ together with the nitrogen atom to which they are attached form a 5-7 membered heterocycloalkyl ring optionally having 1 additional ring heteroatom selected from O, S, and N, wherein the heterocycloalkyl ring is substituted with —CF₃ or —Y₁—X—Y₂—Z;

R₃ is H, or R₃ and R₄ together with the carbon atom to which they are attached form a 5-7 membered heterocycloalkyl ring having 1-2 ring heteroatoms selected from O, S, and N, wherein the heterocycloalkyl ring is optionally fused to a C₅₋₁₀ aryl ring;

R₅ is H, or R₅ and R₄ together with the carbon atoms to which they are attached form a C₆₋₁₂ cycloalkyl, wherein the cycloalkyl ring is optionally fused to a 3-5 membered heterocycloalkyl ring having 1-2 ring heteroatoms selected from O, S, and N, and the cycloalkyl ring is optionally substituted with 1-2 R₇;

wherein one of R₃ and R₅ is not H;

R₆ is H;

each R₇ is independently H or C₁₋₆ alkyl;

each of Y₁ and Y₂ is independently a bond, —NR₇—, or —C(O)NR₇—;

X is 5-6 membered heteroaryl having 1-2 ring heteroatoms selected from O, S, and N, or C₆₋₁₀ aryl;

Z is 6-10 membered heteroaryl having 1-3 ring heteroatoms selected from O, S, and N, 12-14 membered heterocycloalkyl having 1-3 ring heteroatoms selected from O, S, and N, or C₆₋₁₀ aryl, each substituted with 1-4 R₈; and

each R₈ is independently halo, OH, C₁₋₆ alkyl, C₅₋₆ cycloalkyl, C(O)NH₂, or C(O)CH₃. In embodiments, each of R₁ and R₂ is independently C₁₋₆ alkyl, or R₁ and R₂ together with the nitrogen atom to which they are attached form a 5-7 membered heterocycloalkyl ring optionally having 1 additional ring heteroatom selected from O, S, and N, wherein the heterocycloalkyl ring is substituted with —CF₃ or —Y₁—X—Y₂—Z;

R₃ is H, or R₃ and R₄ together with the carbon atom to which they are attached form a 5-7 membered heterocycloalkyl ring having 1-2 ring heteroatoms selected from O, S, and N, wherein the heterocycloalkyl ring is optionally fused to a C₅₋₁₀ aryl ring;

R₅ is H, or R₅ and R₄ together with the carbon atoms to which they are attached form a C₆₋₁₂ cycloalkyl, wherein the cycloalkyl ring is optionally fused to a 3-5 membered heterocycloalkyl ring having 1-2 ring heteroatoms selected from O, S, and N, and the cycloalkyl ring is optionally substituted with 1-2 R₇;

wherein one of R₃ and R₅ is not H;

R₆ is H;

each R₇ is independently H or C₁₋₆ alkyl;

each of Y₁ and Y₂ is independently a bond, —NR₇—, or —C(O)NR₇—;

X is 5-6 membered heteroaryl having 1-2 ring heteroatoms selected from O, S, and N;

Z is 6-10 membered heteroaryl having 1-3 ring heteroatoms selected from O, S, and N, or C₅₋₁₀ aryl, each substituted with 1-4 R₈; and

each R₈ is independently halo, OH, C₁₋₆ alkyl, C₅₋₆ cycloalkyl, or C(O)CH₃.

In various embodiments, each of R₁ and R₂ is independently C₁₋₆ alkyl. In embodiments, each of R₁ and R₂ is independently methyl. In embodiments, one of R₁ and R₂ is C₁₋₆ alkyl and the other is C₁₋₆ alkylene-Y₁—X—Y₂—Z. In embodiments, R₁ is methyl and R₂ is C₁₋₆ alkylene-Y₁—X—Y₂—Z.

In various embodiments, R₁ and R₂ together with the nitrogen atom to which they are attached form a 5-7 membered heterocycloalkyl ring optionally having 1 additional ring heteroatom selected from O, S, and N, wherein the heterocycloalkyl ring is substituted with —CF₃ or —Y₁—X—Y₂—Z. In embodiments, R₁ and R₂ together with the nitrogen atom to which they are attached form a 6 membered heterocycloalkyl ring optionally having 1 additional ring N heteroatom. In embodiments, R₁ and R₂ together with the nitrogen atom to which they are attached form a 6 membered heterocycloalkyl ring having 0 additional ring N heteroatoms. In embodiments, R₁ and R₂ together with the nitrogen atom to which they are attached form a 6 membered heterocycloalkyl ring having 1 additional ring N heteroatom. In embodiments, the heterocycloalkyl ring is substituted with —CF₃. In embodiments, the heterocycloalkyl ring is substituted with —Y₁—X—Y₂—Z.

In various embodiments, R₃ and R₄ together with the carbon atom to which they are attached form a 5-7 membered heterocycloalkyl ring having 1-2 ring heteroatoms selected from O, S, and N, wherein the heterocycloalkyl ring is optionally fused to a C₆₋₁₀ aryl ring, and R₅ is H. In embodiments, R₃ and R₄ together with the carbon atom to which they are attached form a 5 membered heterocycloalkyl ring having 1-2 ring heteroatoms selected from O, S, and N, wherein the heterocycloalkyl ring is optionally fused to a C₆₋₁₀ aryl ring. In embodiments, R₃ and R₄ together with the carbon atom to which they are attached form a 6 membered heterocycloalkyl ring having 1-2 ring heteroatoms selected from O, S, and N, wherein the heterocycloalkyl ring is optionally fused to a C₆₋₁₀ aryl ring. In embodiments, R₃ and R₄ together with the carbon atom to which they are attached form a 7 membered heterocycloalkyl ring having 1-2 ring heteroatoms selected from O, S, and N, wherein the heterocycloalkyl ring is optionally fused to a C₆₋₁₀ aryl ring. In embodiments, R₃ and R₄ together with the carbon atom to which they are attached form a 5 membered heterocycloalkyl ring having 1 ring N heteroatom. In embodiments, R₃ and R₄ together with the carbon atom to which they are attached form a 6 membered heterocycloalkyl ring having 1 ring N heteroatom. In embodiments, R₃ and R₄ together with the carbon atom to which they are attached form a 7 membered heterocycloalkyl ring having 1 ring N heteroatom. In embodiments, R₃ and R₄ together with the carbon atom to which they are attached form a 5-7 membered heterocycloalkyl ring having 1 ring N heteroatom, wherein the heterocycloalkyl ring is fused to a C₆₋₁₀ aryl ring. In embodiments, R₃ and R₄ together with the carbon atom to which they are attached form a 5-7 membered heterocycloalkyl ring having 1 ring N heteroatom, wherein the heterocycloalkyl ring is fused to a phenyl ring. In embodiments, R₃ and R₄ together with the carbon atom to which they are attached form a 5 membered heterocycloalkyl ring having 1 ring N heteroatom, wherein the heterocycloalkyl ring is fused to a phenyl ring. In embodiments, R₃ and R₄ together with the carbon atom to which they are attached form a structure:

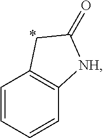

wherein the * indicates the point of attachment to the rest of the molecule. In any of the preceding embodiments, R₅ is H.

In various embodiments, R₅ and R₄ together with the carbon atoms to which they are attached form a C₆₋₁₂ cycloalkyl, wherein the cycloalkyl ring is optionally fused to a 3-5 membered heterocycloalkyl ring having 1-2 ring heteroatoms selected from O, S, and N, and the cycloalkyl ring is optionally substituted with 1-2 R₇, and R₃ is H. In embodiments, R₅ and R₄ together with the carbon atoms to which they are attached form a C₁₀ cycloalkyl, wherein the cycloalkyl ring is optionally fused to a 3-5 membered heterocycloalkyl ring having 1-2 ring heteroatoms selected from O, S, and N. In embodiments, R₅ and R₄ together with the carbon atoms to which they are attached form a C₁₀ cycloalkyl, wherein the cycloalkyl ring is optionally fused to a 3 membered heterocycloalkyl ring having 1 ring O heteroatom. In embodiments, the cycloalkyl ring is unsubstituted. In embodiments, the cycloalkyl ring is substituted with 1-2 R₇. In embodiments, the cycloalkyl ring is substituted with 1 R₇. In embodiments, the cycloalkyl ring is substituted with 2 R₇. In embodiments, each R₇ is independently H. In embodiments, In embodiments, each R₇ is independently C₁₋₆ alkyl. In embodiments, each R₇ is independently methyl. In embodiments, the cycloalkyl ring is substituted with 1 or 2 methyl. In embodiments, the cycloalkyl ring is substituted with 1 methyl. In embodiments, the cycloalkyl ring is substituted with 2 methyl. In embodiments, R₅ and R₄ together with the carbon atoms to which they are attached form a structure:

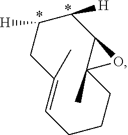

wherein the * each indicate a point of attachment to the rest of the molecule. In any of the preceding embodiments, R₃ is H.

In various embodiments, at least one of Y₁ and Y₂ is —NR₇— or —C(O)NR₇—. In embodiments, one of Y₁ and Y₂ is —NR₇— or —C(O)NR₇—. In embodiments, one of Y₁ and Y₂ is —NR₇— and the other is —C(O)NR₇—. In embodiments, at least one of Y₁ and Y₂ is a bond. In embodiments, one of Y₁ and Y₂ is a bond. In embodiments, Y₁ is a bond. In embodiments, both of Y₁ and Y₂ are bonds. In embodiments, Y₁ is —C(O)NR₇—. In embodiments, Y₁ is —C(O)NH—. In embodiments, Y₂ is —NR₇. In embodiments, Y₂ is —NH. In embodiments, Y₁ is a bond and Y₂ is —NH. In embodiments, both of Y₁ and Y₁ are —C(O)NR₇—. In embodiments, both of Y₁ and Y₁ are —C(O)NH—.

In various embodiments, X is 5-6 membered heteroaryl having 1-2 ring heteroatoms selected from O, S, and N. In embodiments, X is 5 membered heteroaryl having 1-2 ring heteroatoms selected from O, S, and N. In embodiments, X is 6 membered heteroaryl having 1-2 ring heteroatoms selected from O, S, and N. In embodiments, X is 5 membered heteroaryl having 2 ring N heteroatoms or 6 membered heteroaryl having 1 ring N heteroatom. In embodiments, X is 5 membered heteroaryl having 2 ring N heteroatoms. In embodiments, X is 6 membered heteroaryl having 1 ring N heteroatom. In embodiments, X is pyridinyl. In embodiments, X is pyrazolyl. In embodiments, X is C₆₋₁₀ aryl. In embodiments, X is phenyl.

In various embodiments, Z is 6-10 membered heteroaryl having 1-3 ring heteroatoms selected from O, S, and N, or C₅₋₁₀ aryl, each substituted with 1-4 R₈. In embodiments, Z is 6-10 membered heteroaryl having 1-3 ring heteroatoms selected from O, S, and N substituted with 1-4 R₈. In embodiments, Z is C₆₋₁₀ aryl, each substituted with 1-4 R₈. In embodiments, Z is 10 membered heteroaryl having 3 ring N heteroatoms, or phenyl. In embodiments, Z is 10 membered heteroaryl having 3 ring N heteroatoms. In embodiments, Z is phenyl. In embodiments, Z is 12-14 membered heterocycloalkyl having 1-3 ring heteroatoms selected from O, S, and N.

In various embodiments, Z is substituted with 1-4 R₈. In embodiments, Z is substituted with 1 R₈. In embodiments, Z is substituted with 2 R₈. In embodiments, Z is substituted with 3 R₈. In embodiments, Z is substituted with 4 R₈. In embodiments, R₈ is halo, OH, C₁₋₆ alkyl, C₅₋₆ cycloalkyl, or C(O)CH₃. In embodiments, R₈ is halo. In embodiments, R₈ is OH, C₁₋₆ alkyl, C₅₋₆ cycloalkyl, or C(O)CH₃. In embodiments, Z has a structure:

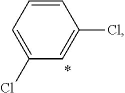

wherein * indicates the point of attachment to the rest of the molecule. In embodiments, Z has a structure:

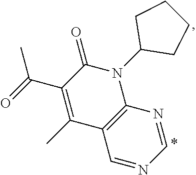

wherein * indicates the point of attachment to the rest of the molecule. In embodiments, Z has a structure:

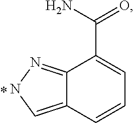

wherein * indicates the point of attachment to the rest of the molecule. In embodiments, Z has a structure:

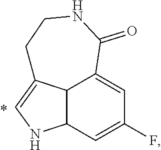

wherein * indicates the point of attachment to the rest of the molecule.

Further provided are compounds as recited in Table 1, or a pharmaceutically acceptable salt thereof. Also provided are use of compounds recited in Table 1, or a pharmaceutically acceptable salt thereof.

TABLE 1

| Compound | |
|---|---|
| # | Structure |
| 19-AP1 | 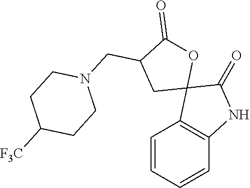 [SMILES: O=C1OC2(CC1CN1CCC(C(F)(F)F)CC1)C(=O)Nc1ccccc12] |
| 19-AP2 | 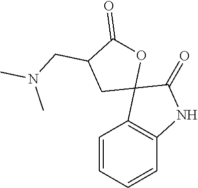 [SMILES: CN(C)CC1CC2(OC1=O)C(=O)Nc1ccccc12] |
| 4 | 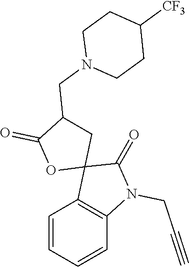 [SMILES: C#CCN1C(=O)C2(CC(CN3CCC(C(F)(F)F)CC3)C(=O)O2)c2ccccc21] |
| 5 | 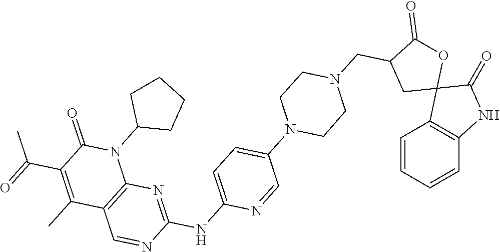 [SMILES: CC(=O)c1c(C)c2cnc(Nc3ccc(N4CCN(CC5CC6(OC5=O)C(=O)Nc5ccccc56)CC4)cn3)nc2n(C2CCCC2)c1=O] |
| 6 | 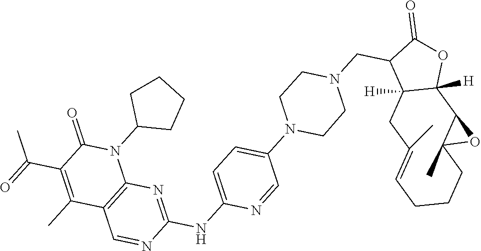 [SMILES: CC(=O)c1c(C)c2cnc(Nc3ccc(N4CCN(CC5C(=O)O[C@@H]6[C@H]7O[C@]7(C)CCC/C=C(\C)C[C@@H]56)CC4)cn3)nc2n(C2CCCC2)c1=O] |
| 7 | 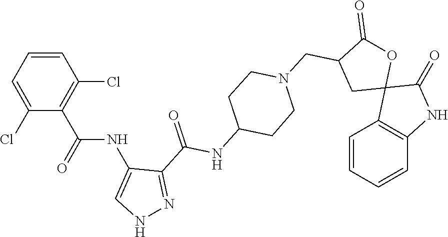 [SMILES: O=C(NC1CCN(CC2CC3(OC2=O)C(=O)Nc2ccccc23)CC1)c1n[nH]cc1NC(=O)c1c(Cl)cccc1Cl] |
| 8 | 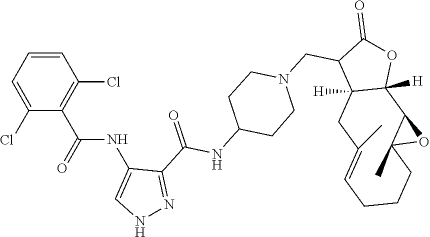 [SMILES: C/C1=C\CCC[C@@]2(C)O[C@@H]2[C@H]2OC(=O)C(CN3CCC(NC(=O)c4n[nH]cc4NC(=O)c4c(Cl)cccc4Cl)CC3)[C@@H]2C1] |
| 9 | 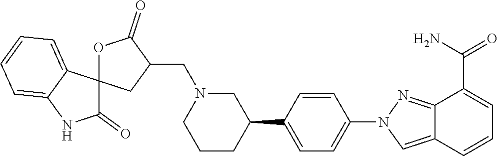 [SMILES: NC(=O)c1cccc2cn(-c3ccc([C@H]4CCCN(CC5CC6(OC5=O)C(=O)Nc5ccccc56)C4)cc3)nc12] |
| 10 | 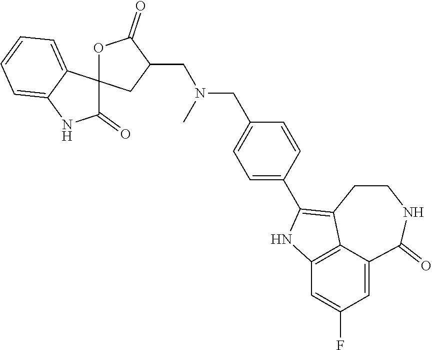 [SMILES: CN(Cc1ccc(-c2[nH]c3cc(F)cc4c3c2CCNC4=O)cc1)CC1CC2(OC1=O)C(=O)Nc1ccccc12] |

In embodiments, the compound is selected from the group consisting of compounds 5, 6, 7, and 8:

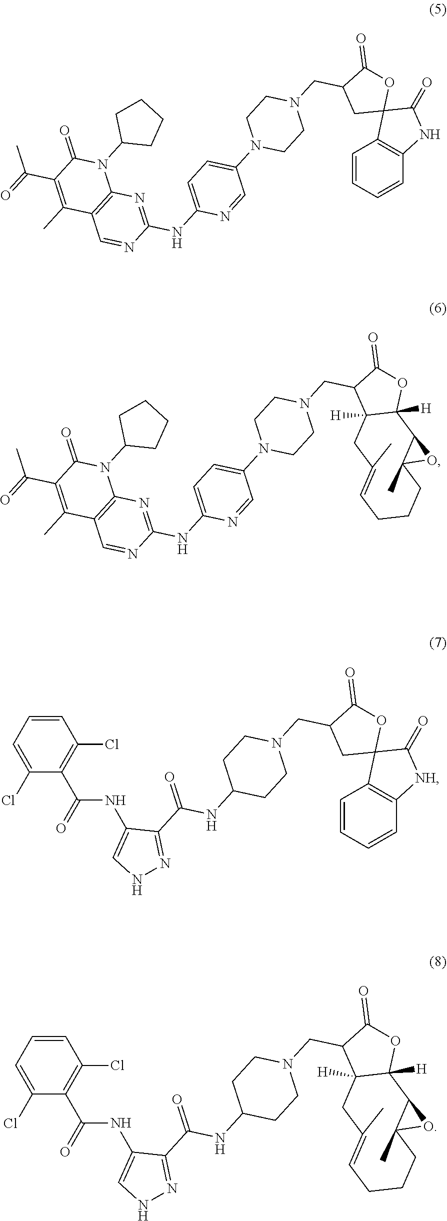 [SMILES: CC(=O)c1c(C)c2cnc(Nc3ccc(N4CCN(CC5CC6(OC5=O)C(=O)Nc5ccccc56)CC4)cn3)nc2n(C2CCCC2)c1=O ; CC(=O)c1c(C)c2cnc(Nc3ccc(N4CCN(CC5C(=O)O[C@@H]6[C@H]7O[C@]7(C)CCC/C=C(\C)C[C@@H]56)CC4)cn3)nc2n(C2CCCC2)c1=O ; O=C(NC1CCN(CC2CC3(OC2=O)C(=O)Nc2ccccc23)CC1)c1n[nH]cc1NC(=O)c1c(Cl)cccc1Cl ; C/C1=C\CCC[C@@]2(C)O[C@@H]2[C@H]2OC(=O)C(CN3CCC(NC(=O)c4n[nH]cc4NC(=O)c4c(Cl)cccc4Cl)CC3)[C@@H]2C1]

The compounds disclosed herein can be in the form of a pharmaceutically acceptable salt. As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al. describe pharmaceutically acceptable salts in detail in J. Pharmaceutical Sciences, 1977, 66, 1-19, which is incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, trifluoroacetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, glutamate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts of compounds containing a carboxylic acid or other acidic functional group can be prepared by reacting with a suitable base. Such salts include, but are not limited to, alkali metal, alkaline earth metal, aluminum salts, ammonium, N*(C₁₋₄alkyl)₄ salts, and salts of organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acids such as lysine and arginine. This invention also envisions the quaternization of any basic nitrogen-containing groups of the compounds disclosed herein. Water or oil-soluble or dispersible products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

### Definitions

As used herein, the term “alkyl” refers to straight chained and branched saturated hydrocarbon groups containing one to thirty carbon atoms, for example, one to twenty carbon atoms, or one to ten carbon atoms. The term Cn means the alkyl group has “n” carbon atoms. For example, C₄ alkyl refers to an alkyl group that has 4 carbon atoms. C₁-C₆ alkyl refers to an alkyl group having a number of carbon atoms encompassing the entire range (e.g., 1 to 6 carbon atoms), as well as all subgroups (e.g., 1-6, 2-7, 1-5, 3-6, 1, 2, 3, 4, 5, and 6 carbon atoms). Nonlimiting examples of alkyl groups include, methyl, ethyl, n-propyl, isopropyl, n-butyl, sec-butyl (2-methylpropyl), and t-butyl. Unless otherwise indicated, an alkyl group can be an unsubstituted alkyl group or a substituted alkyl group.

The term “alkylene” used herein refers to an alkyl group having a substituent. For example, an alkylene group can be —CH₂CH₂— or —CH₂—. The term Cn means the alkylene group has “n” carbon atoms. For example, C₁₋₆ alkylene refers to an alkylene group having a number of carbon atoms encompassing the entire range, as well as all subgroups, as previously described for “alkyl” groups. Unless otherwise indicated, an alkylene group can be an unsubstituted alkylene group or a substituted alkylene group.

As used herein, the term “cycloalkyl” refers to an aliphatic cyclic hydrocarbon group containing five to twelve carbon atoms (e.g., 5, 6, 7, 8, 9, 10, 11, or 12 carbon atoms). The term Cn means the cycloalkyl group has “n” carbon atoms. For example, C₅ cycloalkyl refers to a cycloalkyl group that has 5 carbon atoms in the ring. C₆-C₁₂ cycloalkyl refers to cycloalkyl groups having a number of carbon atoms encompassing the entire range (e.g., 6 to 12 carbon atoms), as well as all subgroups (e.g., 6-7, 6-8, 7-8, 6-9, 7-9, 8-9, 6-10, 7-10, 8-10, 9-10, 6-11, 7-11, 8-11, 9-11, 10-11, 6-12, 7-12, 8-12, 9-12, 10-12, 11-12, 6, 7, 8, 9, 10, 11, and 12 carbon atoms). Nonlimiting examples of cycloalkyl groups include cyclopropyl, cyclobutyl, cyclopentyl, cyclohexyl, cycloheptyl, and cyclooctyl. Unless otherwise indicated, a cycloalkyl group can be an unsubstituted cycloalkyl group or a substituted cycloalkyl group. The cycloalkyl groups described herein can be isolated or fused to another cycloalkyl group, a heterocycloalkyl group, an aryl group and/or a heteroaryl group. When a cycloalkyl group is fused to another cycloalkyl group, then each of the cycloalkyl groups can contain three to twelve carbon atoms unless specified otherwise. Unless otherwise indicated, a cycloalkyl group can be unsubstituted or substituted.

As used herein, the term “heterocycloalkyl” is defined similarly as cycloalkyl, except the ring contains one to three heteroatoms independently selected from oxygen, nitrogen, and sulfur. In particular, the term “heterocycloalkyl” refers to a ring containing a total of three to seven atoms (e.g., three to seven, or five to seven), of which 1, 2, or three of those atoms are heteroatoms independently selected from the group consisting of oxygen, nitrogen, and sulfur, and the remaining atoms in the ring are carbon atoms. Nonlimiting examples of heterocycloalkyl groups include piperdine, pyrazolidine, tetrahydrofuran, tetrahydropyran, dihydrofuran, morpholine, and the like.

Cycloalkyl and heterocycloalkyl groups can be saturated or partially unsaturated ring systems optionally substituted with, for example, one to three groups, independently selected from halo, OH, C(O)—C₁₋₆ alkyl, C(O)NH₂, and C₅₋₆ cycloalkyl. Heterocycloalkyl groups optionally can be further N-substituted with alkyl, alkylene-OH, alkylenearyl, and alkyleneheteroaryl. The heterocycloalkyl groups described herein can be isolated or fused to another heterocycloalkyl group, a cycloalkyl group, an aryl group, and/or a heteroaryl group. When a heterocycloalkyl group is fused to another heterocycloalkyl group, then each of the heterocycloalkyl groups can contain three to fourteen total ring atoms, and one to three heteroatoms, e.g., 12 to 14 total ring atoms, such as 12, 13, or 14 ring atoms, and one to three heteroatoms. Unless otherwise indicated, a heterocycloalkyl group can be unsubstituted or substituted.

As used herein, the term “aryl” refers to a monocyclic aromatic group, such as phenyl. Unless otherwise indicated, an aryl group can be unsubstituted or substituted with one or more, and in particular one to four groups independently selected from, for example, halo, OH, C(O)—C₁₋₆ alkyl, C(O)NH₂, and C₅₋₆ cycloalkyl. Aryl groups can be isolated (e.g., phenyl) or fused to another aryl group (e.g., naphthyl, anthracenyl), a cycloalkyl group (e.g. tetraydronaphthyl), a heterocycloalkyl group, and/or a heteroaryl group. Exemplary aryl groups include, but are not limited to, phenyl, chlorophenyl, methylphenyl, methoxyphenyl, trifluoromethylphenyl, nitrophenyl, 2,4-methoxychlorophenyl, and the like.

As used herein, the term “heteroaryl” refers to a monocyclic or bicyclic aromatic ring having 5 to 10 total ring atoms, and containing one to four heteroatoms selected from nitrogen, oxygen, and sulfur atom in the aromatic ring. Unless otherwise indicated, a heteroaryl group can be unsubstituted or substituted with one or more, and in particular one to four, substituents selected from, for example, halo, OH, C(O)—C₁₋₆ alkyl, C(O)NH₂, and C₅₋₆ cycloalkyl. In embodiments, the heteroaryl group is substituted with one or more of alkyl and alkoxy groups. Examples of heteroaryl groups include, but are not limited to, thienyl, furyl, pyridyl, pyrrolyl, oxazolyl, triazinyl, triazolyl, isothiazolyl, isoxazolyl, imidazolyl, pyrazinyl, pyrimidinyl, thiazolyl, and thiadiazolyl.

As used herein, the term “substituted,” when used to modify a chemical functional group, refers to the replacement of at least one hydrogen radical on the functional group with a substituent. Substituents can include, but are not limited to, alkyl, cycloalkyl, alkenyl, cycloalkenyl, alkynyl, heterocycloalkyl, aryl, heteroaryl, hydroxyl, oxy, alkoxy, heteroalkoxy, ester, thioester, carboxy, cyano, nitro, amino, amido, acetamide, and halo (e.g., fluoro, chloro, bromo, or iodo). When a chemical functional group includes more than one substituent, the substituents can be bound to the same carbon atom or to two or more different carbon atoms.

As used herein, the phrase “optionally substituted” means unsubstituted (e.g., substituted with a H) or substituted. As used herein, the term “substituted” means that a hydrogen atom is removed and replaced by a substituent. It is understood that substitution at a given atom is limited by valency. The use of a substituent (radical) prefix name such as alkyl without the modifier “optionally substituted” or “substituted” is understood to mean that the particular substituent is unsubstituted.

As used herein, the term “therapeutically effective amount” means an amount of a compound or combination of therapeutically active compounds (e.g., a NFκB modulator or combination of NFκB modulators) that ameliorates, attenuates or eliminates one or more symptoms of a particular disease or condition (e.g., cancer), or prevents or delays the onset of one of more symptoms of a particular disease or condition.

As used herein, the terms “patient” and “subject” may be used interchangeably and mean animals, such as dogs, cats, cows, horses, and sheep (e.g., non-human animals) and humans. Particular patients or subjects are mammals (e.g., humans). The terms patient and subject include males and females.

As used herein, the term “pharmaceutically acceptable” means that the referenced substance, such as a compound of the present disclosure, or a formulation containing the compound, or a particular excipient, are safe and suitable for administration to a patient or subject. The term “pharmaceutically acceptable excipient” refers to a medium that does not interfere with the effectiveness of the biological activity of the active ingredient(s) and is not toxic to the host to which it is administered.

As used herein the terms “treating”, “treat” or “treatment” and the like include preventative (e.g., prophylactic) and palliative treatment.

As used herein, the term “excipient” means any pharmaceutically acceptable additive, carrier, diluent, adjuvant, or other ingredient, other than the active pharmaceutical ingredient (API).

### Synthesis of Compounds of the Disclosure

The compounds disclosed herein can be prepared in a variety of ways using commercially available starting materials, compounds known in the literature, or from readily prepared intermediates, by employing standard synthetic methods and procedures either known to those skilled in the art, or in light of the teachings herein. Standard synthetic methods and procedures for the preparation of organic molecules and functional group transformations and manipulations can be obtained from the relevant scientific literature or from standard textbooks in the field. The following descriptions of synthetic methods are designed to illustrate, but not to limit, general procedures for the preparation of compounds of the present disclosure.

The synthetic processes disclosed herein can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt, ester or prodrug thereof.

Synthetic procedures for preparing the compounds disclosed herein can be found in the Examples section.

### Pharmaceutical Formulations, Dosing, and Routes of Administration

Further provided are pharmaceutical formulations comprising a compound as described herein (e.g., compounds of Formula I, Table 1, or pharmaceutically acceptable salts of the compounds) and a pharmaceutically acceptable excipient.

The compounds described herein can be administered to a subject in a therapeutically effective amount (e.g., in an amount sufficient to prevent or relieve the symptoms of a cancer). The compounds can be administered alone or as part of a pharmaceutically acceptable composition or formulation. In addition, the compounds can be administered all at once, multiple times, or delivered substantially uniformly over a period of time. It is also noted that the dose of the compound can be varied over time.

A particular administration regimen for a particular subject will depend, in part, upon the compound, the amount of compound administered, the route of administration, and the cause and extent of any side effects. The amount of compound administered to a subject (e.g., a mammal, such as a human) in accordance with the disclosure should be sufficient to effect the desired response over a reasonable time frame. Dosage typically depends upon the route, timing, and frequency of administration. Accordingly, the clinician titers the dosage and modifies the route of administration to obtain the optimal therapeutic effect, and conventional range-finding techniques are known to those of ordinary skill in the art.

Purely by way of illustration, the method can include administering, e.g., from about 0.1 mg/kg up to about 100 mg/kg of compound or more, depending on the factors mentioned above. In other embodiments, the dosage ranges from 1 mg/kg up to about 100 mg/kg; or 5 mg/kg up to about 100 mg/kg; or 10 mg/kg up to about 100 mg/kg. Some conditions require prolonged treatment, which may or may not entail administering lower doses of compound over multiple administrations. If desired, a dose of the compound is administered as two, three, four, five, six or more sub-doses administered separately at appropriate intervals throughout the day, optionally, in unit dosage forms. The treatment period will depend on the particular condition and type of pain, and may last one day to several months.

Suitable methods of administering a physiologically-acceptable composition, such as a pharmaceutical composition comprising the compounds disclosed herein (e.g., compounds of Formula I or Table 1), are well known in the art. Although more than one route can be used to administer a compound, a particular route can provide a more immediate and more effective reaction than another route. Depending on the circumstances, a pharmaceutical composition comprising the compound is applied or instilled into body cavities, absorbed through the skin or mucous membranes, ingested, inhaled, and/or introduced into circulation. For example, in certain circumstances, it will be desirable to deliver a pharmaceutical composition comprising the agent orally, parenterally, through injection by intravenous, intraperitoneal, intracerebral (intra-parenchymal), epidural, intracerebroventricular, intramuscular, intra-ocular, intraarterial, intracarotid, intraportal, intralesional, intramedullary, intrathecal, intraventricular, transdermal, subcutaneous, intraperitoneal, intranasal, pulmonary, enteral, topical, intradermal, sublingual, urethral, vaginal, or rectal means, by sustained release systems, or by implantation devices. If desired, the compound is administered regionally via intrathecal administration, intracerebral (intra-parenchymal) administration, intracerebroventricular administration, or intraarterial or intravenous administration feeding the region of interest. Alternatively, the composition is administered locally via implantation of a membrane, sponge, or another appropriate material onto which the desired compound has been absorbed or encapsulated. Where an implantation device is used, the device is, in one aspect, implanted into any suitable tissue or organ, and delivery of the desired compound is, for example, via diffusion, timed-release bolus, or continuous administration.

To facilitate administration, the compound is, in various aspects, formulated into a physiologically-acceptable composition comprising a carrier (e.g., vehicle, adjuvant, or diluent). The particular carrier employed is limited only by chemico-physical considerations, such as solubility and lack of reactivity with the compound, and by the route of administration. Physiologically-acceptable carriers are well known in the art. Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). Injectable formulations are further described in, e.g., Pharmaceutics and Pharmacy Practice, J. B. Lippincott Co., Philadelphia. Pa., Banker and Chalmers, eds., pages 238-250 (1982), and ASHP Handbook on Injectable Drugs, Toissel, 4th ed., pages 622-630 (1986)). A pharmaceutical composition comprising the compound is, in one aspect, placed within containers, along with packaging material that provides instructions regarding the use of such pharmaceutical compositions. Generally, such instructions include a tangible expression describing the reagent concentration, as well as, in certain embodiments, relative amounts of excipient ingredients or diluents (e.g., water, saline or PBS) that may be necessary to reconstitute the pharmaceutical composition.

Compositions suitable for parenteral injection may comprise physiologically acceptable sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents, or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

These compositions may also contain adjuvants such as preserving, wetting, emulsifying, and dispersing agents. Microorganism contamination can be prevented by adding various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents delaying absorption, for example, aluminum monostearate and gelatin.

Solid dosage forms for oral administration include capsules, tablets, powders, and granules. In such solid dosage forms, the active compound is admixed with at least one inert customary excipient (or carrier) such as sodium citrate or dicalcium phosphate or (a) fillers or extenders, as for example, starches, lactose, sucrose, mannitol, and silicic acid; (b) binders, as for example, carboxymethylcellulose, alginates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (a) solution retarders, as for example, paraffin; (f) absorption accelerators, as for example, quaternary ammonium compounds; (g) wetting agents, as for example, cetyl alcohol and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, and tablets, the dosage forms may also comprise buffering agents. Solid compositions of a similar type may also be used as fillers in soft and hard filled gelatin capsules using such excipients as lactose or milk sugar, as well as high molecular weight polyethylene glycols, and the like.

Solid dosage forms such as tablets, dragees, capsules, pills, and granules can be prepared with coatings and shells, such as enteric coatings and others well known in the art. The solid dosage forms may also contain opacifying agents. Further, the solid dosage forms may be embedding compositions, such that they release the active compound or compounds in a certain part of the intestinal tract in a delayed manner. Examples of embedding compositions that can be used are polymeric substances and waxes. The active compound can also be in micro-encapsulated form, optionally with one or more excipients.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil, and sesame seed oil, glycerol, tetrahydrofurfuryl alcohol, polyethylene glycols and fatty acid esters of sorbitan, or mixtures of these substances, and the like.

Besides such inert diluents, the composition can also include adjuvants, such as wetting agents, emulsifying and suspending agents, sweetening, flavoring, and perfuming agents. Suspensions, in addition to the active compound, may contain suspending agents, as for example, ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

Compositions for rectal administration are preferably suppositories, which can be prepared by mixing the compounds of the disclosure with suitable non-irritating excipients or carriers such as cocoa butter, polyethylene glycol or a suppository wax, which are solid at ordinary room temperature, but liquid at body temperature, and therefore, melt in the rectum or vaginal cavity and release the active component.

The compositions used in the methods of the invention may be formulated in micelles or liposomes. Such formulations include sterically stabilized micelles or liposomes and sterically stabilized mixed micelles or liposomes. Such formulations can facilitate intracellular delivery, since lipid bilayers of liposomes and micelles are known to fuse with the plasma membrane of cells and deliver entrapped contents into the intracellular compartment.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

The frequency of dosing will depend on the pharmacokinetic parameters of the agents and the routes of administration. The optimal pharmaceutical formulation will be determined by one of skill in the art depending on the route of administration and the desired dosage. See, for example, Remington's Pharmaceutical Sciences, 18th Ed. (1990) Mack Publishing Co., Easton, Pa., pages 1435-1712, incorporated herein by reference. Such formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein, as well as the pharmacokinetic data observed in animals or human clinical trials.

The precise dosage to be employed depends upon several factors including the host, whether in veterinary medicine or human medicine, the nature and severity of the condition, e.g., disease or disorder, being treated, the mode of administration and the particular active substance employed. The compounds may be administered by any conventional route, in particular enterally, and, in one aspect, orally in the form of tablets or capsules. Administered compounds can be in the free form or pharmaceutically acceptable salt form as appropriate, for use as a pharmaceutical, particularly for use in the prophylactic or curative treatment of a disease of interest. These measures will slow the rate of progress of the disease state and assist the body in reversing the process direction in a natural manner.

It will be appreciated that the pharmaceutical compositions and treatment methods of the invention are useful in fields of human medicine and veterinary medicine. Thus the subject to be treated is in one aspect a mammal. In another aspect, the mammal is a human.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

### METHODS OF USE

The compositions of the present invention can be used to treat and/or prevent a variety of diseases. In one embodiment the disease is a form of cancer. In a specific embodiment the cancer is ovarian cancer. In one embodiment the compositions of the present invention can be administered with at least one other therapeutic agent (e.g. other anti-cancer agents).

Although significant advancements have been made to treat cancer, ovarian cancer remains the leading cause of gynecological cancer deaths in the United States. Several reports show that NFκB pathway is dysregulated in ovarian cancer. In a panel of ovarian cancer cell lines, we observe elevated levels of RELA when compared to immortalized fallopian tube epithelial cell lines, which are an appropriate normal control for most ovarian cancers. Consistently, analyses of the TCGA data from 17 cancer types show that RELA (p65) expression is the highest in ovarian tumors. These suggest that targeting the NFκB pathway proteins (RELA and IKKα) is a viable therapeutic strategy for ovarian cancer. Moreover, CDK4/6 has been implicated in ovarian cancer tumorigenesis and resistance to therapy.

The compounds described herein (e.g., the compounds of Formula I or compounds of Table 1) can inhibit an NFκB pathway. The compounds disclosed herein are particularly advantageous for the treatment of diseases or disorders caused by aberrant expression or activity of an NFκB pathway. The incidence and/or intensity of diseases or disorders associated with aberrant expression or activity of an NFκB pathway is reduced.

Increased expression and/or activity of an NFκB pathway includes overexpression or hyperactivity of any component of an NFκB pathway. Overexpression and/or hyperactivity of the NFκB pathways is well known to cause many adverse conditions. These include, for example, cancer, autoimmune diseases, inflammatory diseases, diabetes, cardiovascular diseases, and neurological diseases. Cancer includes but is not limited to ovarian cancer, breast cancer, prostate cancer, colon cancer, liver cancer, brain cancer, kidney cancer, lung cancer, leukemia, lymphoma, multiple myeloma, thyroid cancer, bone cancer, esophageal cancer, and pancreatic cancer. Inflammatory diseases include but are not limited to arthritis, rheumatoid arthritis, atherosclerosis, multiple sclerosis, asthma, inflammatory bowel disease, Crohn's disease, gastritis, pancreatitis, systemic inflammatory response syndrome, and chronic inflammatory demyelinating polyradiculoneuritis.

NFκB selective inhibitors can be used for cancer prevention and treatment. The relationship between NFκB activation and inflammation-associated cancer have been demonstrated using several mouse models. NFκB activation has been implicated in inflammation associated liver, prostate and colon cancer induction in humans and mouse models. Several antioxidants having electrophilic capacity such as cyclopentenone prostaglandins, dimethoxylsulfoxide, glutathione and non-steroidal anti-inflammatory drugs (NSAIDs) Ibuprofen, sulindac, as well as curcumin inhibit NFκB activity but do not show high selectivity. Aspirin, sulfasalazine, SC-514, and PS-1145 also inhibit NFκB by interrupting phosphorylation of IKK.

Compounds of Formula I and Table 1 display high selectivity for growth inhibition and/or induction of apoptosis in cancer cells, e.g., in ovarian cancer cells.

The disclosed methods include methods for treating disease or disorder capable of being modulated by inhibition of the NFκB pathway, e.g., cancer, comprising administering to a subject a compound that binds a component of the NFκB pathway. In some examples, the compound disrupts binding of a protein which activates the NFκB pathway. In one example, the method includes use of a compound that disrupts binding of a protein to TNFα. In another example, the method includes use of a compound that disrupts binding of a protein to IKKβ. In another example, the compound prevents translocation of NFκB to the nucleus.

Provided herein is a method of modulating the NFκB pathway in a cell, comprising contacting the cell with a compound or a composition as disclosed herein (e.g., the compounds of Formula I or as shown in Table 1) in an amount sufficient to modulate the NFκB pathway. The contacting of the cell can occur in vitro or in vivo. In some embodiments, contacting of the cell occurs in vitro. In other embodiments, contacting of the cell occurs in vivo. Therefore, the disclosure includes administering one or more of a compound described herein to a subject, such as a human, in need thereof. In some embodiments, the subject suffers from a disease or disorder associated with aberrant activity of the NFκB pathway. Disorders associated with aberrant activity of the NFκB pathway include, but are not limited to, cancer (e.g., ovarian cancer), autoimmune diseases, inflammatory diseases, diabetes, cardiovascular diseases, and neurological diseases. Specifically contemplated cancers include ovarian cancer, breast cancer, prostate cancer, colon cancer, liver cancer, brain cancer, kidney cancer, lung cancer, leukemia, lymphoma, multiple myeloma, thyroid cancer, bone cancer, esophageal cancer, and pancreatic cancer.

The disclosed methods utilize compounds that inhibit the NFκB pathway, for treating, e.g., cancer. Methods for assessing the usefulness of a compound for treating cancer are known to those of skill in the art. For example, compounds may be assessed using models of cancer, including cells (such as ovarian cancer cells), animal models (such as mouse xenograph or other cancer models), or in human subjects having, e.g., ovarian cancer.

The compounds described herein can be used to decrease or prevent cancer in human subjects with e.g., ovarian cancer. In a particular example, a compound or mixture is administered orally, such as by mixing with distilled water. In another example, a test compound or mixture is administered intravenously, such as in saline or distilled water. In some examples, treatment with test compound may be a single dose or repeated doses. The test compound may be administered about every 6 hours, about every 12 hours, about every 24 hours (daily), about every 48 hours, about every 72 hours, or about weekly. Treatment with repeated doses may continue for a period of time, for example for about 1 week to 12 months, such as about 1 week to about 6 months, or about 2 weeks to about 3 months, or about 1 to 2 months. Administration of a compound may also continue indefinitely. Doses of test compound are from about 0.1 mg/kg to about 400 mg/kg, such as about 1 mg/kg to about 300 mg/kg, about 2 mg/kg to 200 mg/kg, about 10 mg/kg to about 100 mg/kg, about 20 mg/kg to about 75 mg/kg, or about 25 mg/kg to about 50 mg/kg.

It will be understood that the methods and compositions described herein for treating cancer, comprising administering a compound that inhibits the NFκB pathway, are applicable to methods of treating other diseases related to NFκB activity, such as those described above. The methods for assessing the effectiveness of test compounds for treating such diseases in cells, appropriate animal models, or affected subjects are known to one of skill in the art.

Uses of the compounds disclosed herein in the preparation of a medicament for treating diseases or disorders related to NFκB activity also are provided herein.

The disclosure herein will be understood more readily by reference to the following examples, below.

### EXAMPLES

The following examples are provided for illustration and are not intended to limit the scope of the disclosure.

### Compound Synthetic Procedures

General Experimental Procedures. All reagents were purchased from commercial sources and were used without further purification. Flash chromatography was carried out on silica gel (200-400 mesh). Thin layer chromatography (TLC) were run on pre-coated EMD silica gel 60 F254 plates and observed under UV light at 254 nm and with basic potassium permanganate dip. Column chromatography was performed with silica gel (230-400 mesh, grade 60, Fisher scientific, USA). Preparative HPLC was carried out on 250×21.2 mm C-18 column using gradient conditions (10-100% B, flow rate=6.0 mL/min, 39 min). The eluents used were: solvent A (H₂O with 0.1% Formic acid) and solvent B (CH₃CN with 0.1% Formic acid).

### Example 1: 4-((4-(trifluoromethyl)piperidin-1-yl)methyl)-3,4-dihydro-5H-spiro[furan-2,3′-indoline]-2′,5dione (Compound 19-AP1)

4-(trifluoromethyl)piperidine (30 mg, 0.20 mmol) and TEA (50 μL) were added sequentially to a solution of 4-methylene-1′-(prop-2-yn-1-yl)-3,4-dihydro-5H-spiro[furan-2,3′indoline]-2′,5-dione (21 mg, 0.10 mmol) in ethanol (2 mL) at RT. The resulting solution was stirred for 3 h. Then the solvent was evaporated to afford the crude residue, which was purified by column chromatography to afford the desired compound as a mixture of diastereomers. C₁₈H₁₉F₃N₂O₃[M]⁺: 368.14; found 369.75.

### Example 2: 4-((dimethylamino)methyl)-3,4-dihydro-5H-spiro[furan-2,3′-indoline]-2′,5-dione (Compound 19-AP2)

To a solution of 4-methylene-1′-(prop-2-yn-1-yl)-3,4-dihydro-5H-spiro[furan-2,3′indoline]-2′,5-dione (60 mg, 0.28 mmol) in ethanol (3 mL) were added dimethylamine hydrochloride (45 mg, 0.56 mmol) and TEA (98 μL). The resulting solution was stirred for 4 h and progress of the reaction was monitored by TLC. The solvent was evaporated to afford the crude residue, which was purified by column chromatography to afford the desired compound as a mixture of diastereomers. C₁₄H₁₆N₂O₃ [M]⁺: 260.12; found 260.99.

### Example 3: 1′-(prop-2-yn-1-yl)-4-((4-(trifluoromethyl)piperidin-1-yl)methyl)-3,4-dihydro-5H-spiro[furan2,3′-indoline]-2′,5-dione (Compound 4)

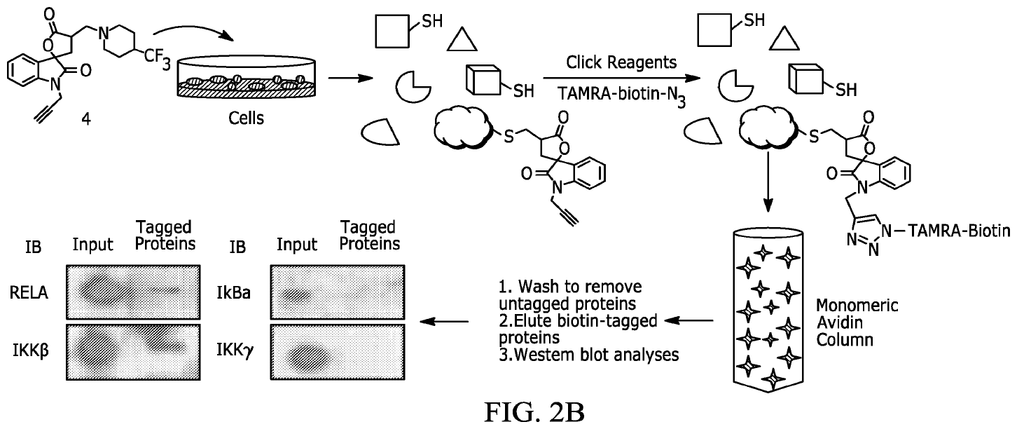

FIG. 2A summarizes the synthesis of compound 4, which is an alkyne-tagged prodrug and an analog of compound 19-AP1. Compound 4 is suitable for in situ click chemistry. Briefly, commercially available isatin (Compound 1) was stirred with propargyl bromide and potassium carbonate in DMF to yield substituted isatin-1-(prop-2-yn-1-yl)indoline-2,3-dione (Compound 2). The intermediate (Compound 2) was subjected to Indium catalyzed Barbier-type reaction followed by an acid-catalyzed cyclization to yield compound 3. (Rana & Natarajan, 2013) Treatment of compound 3 with 4-trifluoromethylpiperidine and triethylamine in methanol resulted in desired alkyne-tagged compound 4.

In particular, to a solution of isatin (200 mg, 1.36 mmol) in anhydrous DMF (6 mL) cooled to 0° C. under argon atmosphere was added 60% NaH (82 mg, 2.04 mmol). After 10 min, propargyl bromide (0.19 mL, 2.04 mmol, 80%) was added at −0° C., and the solution was allowed to warm to ambient temperature and stirred for 16 h. Reaction mixture was washed with NH₄Cl and extracted with EtOAc. The organic phase was washed with brine, separated, dried over MgSO₄, and the solvent was evaporated in vacuo. The crude residue was purified by silica gel chromatography to afford the pure product. C₁₁H₇NO₂ [M]⁺: 185.05; found 186.27.

In a round bottom flask, 1-(prop-2-yn-1-yl)indoline-2,3-dione (40 mg, 0.22 mmol) was dissolved in THF:water (2 ml, 2:1) followed by addition of Indium powder (49 mg, 0.43 mmol) and methyl 2-(bromomethyl) acrylate (77 mg, 0.43 mmol). Reaction was stirred at room temperature for 24 h. Crude was dissolved in ethyl acetate and wash 1N HCl, and brine.

The crude mixture was purified via column chromatography using hexane and ethyl acetate gradient to obtain methyl 2-((3-hydroxy-2-oxo-1-(prop-2-yn-1-yl)indolin-3-yl)methyl)acrylate (intermediate 1). C₁₆H₁₅NO₄ [M]⁺: 285.10; found 286.07.

Methyl 2-((3-hydroxy-2-oxo-1-(prop-2-yn-1-yl)indolin-3-yl)methyl)acrylate (Intermediate 1, 33 mg, 0.12 mmol) was taken in 3 mL of DCM and cooled to 0° C. followed by addition of p-toluene sulfonic acid (45 mg, 0.24 mmol) Crude mixture was diluted in ethyl acetate and washed with brine, dried MgSO₄, and purified by column chromatography to yield 4-methylene-1′-(prop-2-yn-1-yl)-3,4-dihydro-5Hspiro[furan-2,3′-indoline]-2′,5-dione (compound 3).

4-(trifluoromethyl)piperidine (9 mL, 0.058 mmol) and TEA (20 mL) were added sequentially to a solution of 4-methylene-1′-(prop-2-yn-1-yl)-3,4-dihydro-5H-spiro[furan-2,3′indoline]-2′,5-dione (Compound 3, 11 mg, 0.038 mmol) in methanol (1 mL) at RT. The resulting solution was stirred for 4 h. Then the solvent was evaporated to afford the crude residue, which was purified by column chromatography to afford compound 4 as a mixture of diastereomers. UPLC-MS calculated for C₂₁H₂₁F₃N₂O₃[M]⁺: 406.41; found 406.79. Compound 4 is an alkyne-tagged analog of compound 19-AP1.

### Example 4: 4-((4-(6-((6-Acetyl-8-cyclopentyl-5-methyl-7-oxo-7,8-dihydropyrido[2,3-d]pyrimidin-2yl)amino)pyridin-3-yl)piperazin-1-yl)methyl)-3,4-dihydro-5H-spiro[furan-2,3′-indoline]-2′,5dione (Compound 5)

To a solution of 4-methylene-3,4-dihydro-5H-spiro[furan-2,3′-indoline]-2′,5-dione (20 mg, 0.093 mmol), triethylamine in methanol (2 mL) was added palbociclib (42 mg, 0.093 mmol) and the mixture was stirred for 48 h. Then the mixture was concentrated in vacuo and purified by prep HPLC. UPLC-MS calculated for C₃₆H₃₈N₈O₅ [M]⁺: 662.29; found 663.30.

### Example 5: 6-Acetyl-8-cyclopentyl-2-((5-(4-(((3R,3aS,9aR,10aS,10bS,E)-6,9a-dimethyl-2-oxo2,3,3a,4,5,8,9,9a,10a,10b-decahydrooxireno[2′,3′:9,10]cyclodeca[1,2-b]furan-3yl)methyl)piperazin-1-yl)pyridin-2-yl)amino)-5-methylpyrido[2,3-d]pyrimidin-7(8H)-one (Compound 6)

To a solution of parthenolide (25 mg, 0.100 mmol), triethylamine in methanol (2 mL) was added palbociclib (45 mg, 0.100 mmol) and the mixture was stirred for 48 h. Then the mixture was concentrated in vacuo and purified by prep HPLC. UPLC-MS calculated for C₃₉H₄₉N₇O₅ [M]⁺: 695.38; found 696.38.

### Example 6: 4-(2,6-dichlorobenzamido)-N-(1-((2′,5-dioxo-4,5-dihydro-3H-spiro[furan-2,3′-indolin]-4yl)methyl)piperidin-4-yl)-1H-pyrazole-3-carboxamide (Compound 7)

To a solution of 4-methylene-3,4-dihydro-5H-spiro[furan-2,3′-indoline]-2′,5-dione (16 mg, 0.074 mmol), triethylamine in methanol (2 mL) was added AT7519 (31 mg, 0.074 mmol) and the mixture was stirred for 48 h. Then the mixture was concentrated in vacuo and purified by prep HPLC. LC-MS calculated for C₂₈H₂₆Cl₂N₆O₅ [M]⁺: 596.13; found 597.2.

### Example 7: 4-(2,6-dichlorobenzamido)-N-(1-(((3R,3aS,9aR,10aS,10bS,E)-6,9a-dimethyl-2-oxo2,3,3a,4,5,8,9,9a,10a,10b-decahydrooxireno[2′,3′:9,10]cyclodeca[1,2-b]furan-3yl)methyl)piperidin-4-yl)-1H-pyrazole-3-carboxamide (Compound 8)

To a solution of parthenolide (16 mg, 0.064 mmol), triethylamine (20 μL) in methanol (2 mL) was added AT7519 (30 mg, 0.070 mmol) and the mixture was stirred for 48 h. Then the mixture was concentrated in vacuo and purified by prep HPLC. LCMS calculated for C₃₁H₃₇Cl₂N₅O₅ [M]⁺: 629.2; found 630.3.

### Biological Assay Data

### Example 8: Stability of Compound 19-AP1

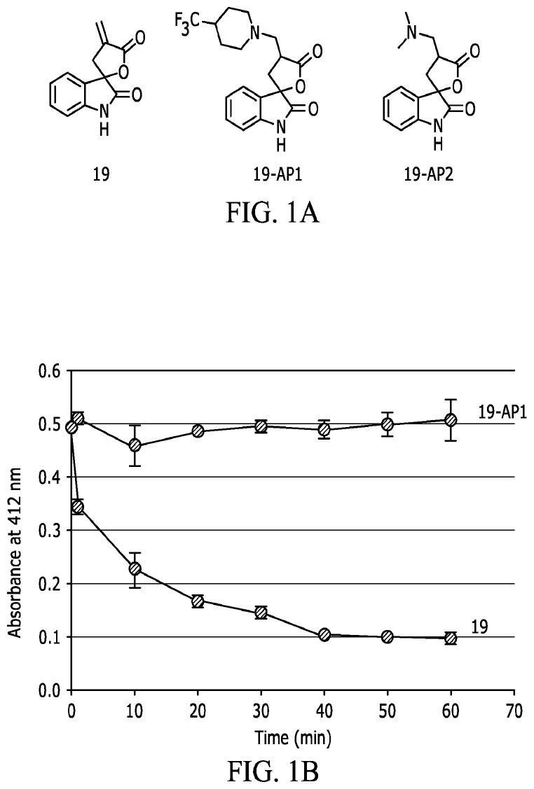

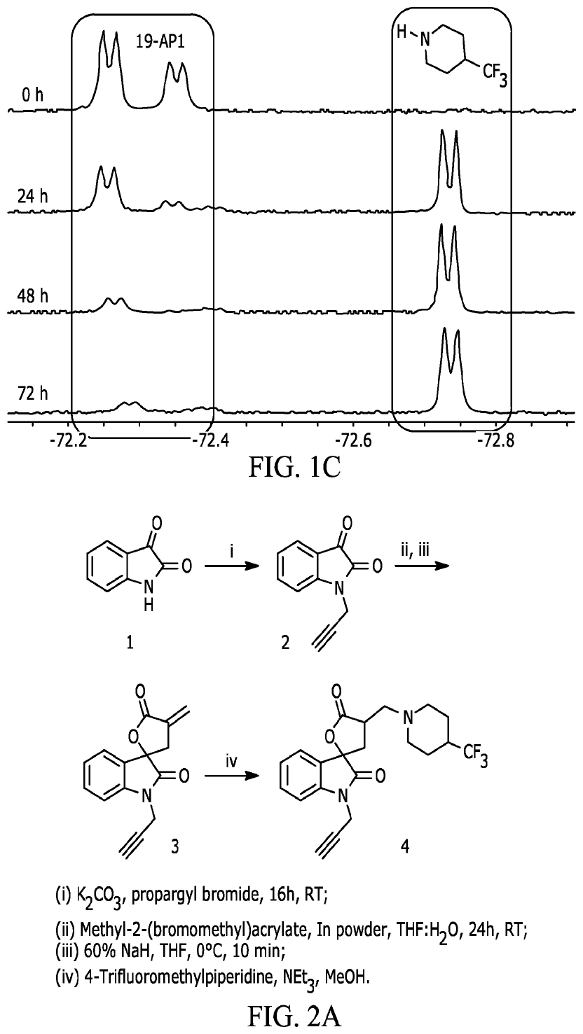

Reaction of 4-methylene-3,4-dihydro-5H-spiro(furan-2,3′-indoline)-2′,5-dione (Compound 19) with 4-trifluoropiperidine in ethanol resulted in the corresponding amino prodrugs 4-((4-(trifluoromethyl)piperidin-1-yl)methyl)-3,4-dihydro-5Hspiro(furan-2,3′-indoline)-2′,5-dione (Compound 19-AP1) as a mixture of diastereomers (FIG. 1A). Without wishing to be bound by theory, it is believed that the α-methylene-γ-butyrolactone moiety on compound 19 is reactive toward biological nucleophiles such as glutathione and cysteine. To determine if secondary amine prodrug of compound 19 (Compound 19-AP1) results in improved stability, the reactivity of analog compound 19 and the prodrug compound 19-AP1 was compared in a cysteine binding assay using Ellman's reagent (FIG. 1B).

Briefly, the cells were incubated with 10 μM of compound 4 for 24 h. In cells, compound 4 is converted to compound 3 with an active methylene group which reacts with surface-exposed cysteine residues on proteins. The lysate was treated with click reagents (TCEP, TBTA and CuSO₄) and azido-biotin. Biotin-tagged-3-bound proteins were subjected to monoavidin column. Biotin-tagged proteins were captured, the column was washed to removed untagged proteins and the biotin-tagged-3-bound proteins were eluted with regeneration buffer (6M urea/PBS). The eluted lysates were subjected to Western blot analyses and probed for proteins (IKKβ, RELA, IκBα and IKKγ) in the IKK complex.

Compound 19 reacted rapidly with free cysteine whereas amine-prodrug compound 19-AP1 is more stable under similar conditions. These studies show that the amine-based prodrugs of α-methylene-γ-butyrolactone containing compounds are stable to biological nucleophiles.

Next, to assess the kinetics of the release of the active compound 19 from the prodrug compound 19-AP1, the release of 4trifluoropiperidine from the prodrug compound 19-AP1 was monitored using ¹⁹F-NMR spectroscopy following a reported method. (Woods, Mo, Bieberich, Alavanja, & Colby, 2011). The ¹⁹F NMR studies showed a time-dependent disappearance of the CF₃ signal on compound 19-AP1 and corresponding appearance of the CF₃ signal on 4-trifluoromethylpiperidine (FIG. 1C). This observation is consistent with previously reported studies with a prodrug of parthenolide. These studies clearly show that compound 19-AP1 is more stable to biological nucleophiles and the prodrug is hydrolyzed slowly to release the active compound 19 over a 72 h period.

### Example 9: Target Engagement Studies of Compounds of the Disclosure with Cellular NFκB Pathway Proteins

To determine target engagement in cells, cancer cells (A2780) were treated with the compound 4, which is an alkyne-tagged analog of prodrug compound 19-AP1 for 48 h (FIG. 2B). Cells were then harvested, washed and pelleted. Unbound compound 4 was removed by extensively washing the cell pellet. The cell pellets were lysed, and 2 mg/mL of lysates were subjected to click chemistry with TAMRA-Biotin-Azide trifunctional probe (10 mM stock, 20 μL, click chemistry tools), and other click reagents (TCEP, 100 mM stock, 20 μL; TBTA 13.5 mM stock, 20 μL and CuSO₄ 100 mM stock, 10 μL) to make total volume of 1 mL. The reaction was incubated for 3 h at RT. The lysates were incubated with monomeric avidin beads for additional 1 h. The sample was washed with 15 mL elution buffer to remove unbound proteins followed by multiple washing with regeneration buffer (15 mL) to cleave biotin-monomeric avidin bond (Pierce® Monomeric Avidin Kit, ThermoScientific, Cat #20227). The collected regeneration sample was washed with an excess of water (50 mL) to remove salts and freeze dried to yield tagged protein powder. Protein powder was dissolved in buffer and subjected to Western blot analyses. The membranes were probed for NFκB pathway proteins IKKβ, RELA, IκBα and IKKγ (FIG. 2B).

Covalent binding of IKKβ, and RELA proteins to compound 4 was observed, but not IκBα or IKKβ proteins despite presence of surface exposed cysteine residues. This is the first report that shows that an amine-based prodrug of α-methylene-γ-butyrolactone containing NFκB inhibitor selectively engaged NFκB pathway proteins RELA and IKKβ in cells. This confirms the release of the parent eneone (Compound 3) from prodrug compound 4, which irreversibly binds to RELA and IKKβ.

### Example 10: Cancer Cell Growth Inhibition

The cancer cell growth-inhibitory potential of amine-prodrugs (compounds 19-AP1 and 19-AP2) was evaluated. Compounds 19, 19-AP1 and 19-AP2 were screened in ovarian cancer lines (A2780, and OVSAHO) and immortalized fallopian tube epithelial cells (FT282E1) in a 72 h growth inhibition assay. In A2780 and FT282E1 cell lines, amine-prodrugs are ˜2-3 fold less active than the parent compound 19 whereas amine-prodrug compounds 19-AP1 and 19-AP2 are more potent in the OVSAHO cell line.

To determine if this difference in activities was due to quenching of the eneone by the thiol nucleophile glutathione (GSH) or release kinetics, cellular GSH levels in the ovarian cancer cell lines were observed. No correlation was observed between the GSH levels and the activity (data summarized in Table 2, below).

TABLE 2

| | IC₅₀ (μM) | | | Glutathione |
|---|---|---|---|---|
| Cell Line | 19 | 19-AP1 | 19-AP2 | (μM/10⁶ cells) |
| A2780 | 2.15 ± 0.15 | 5.71 ± 0.24 | 5.38 ± 0.33 | 1.4 ± 1.7 |
| FT282E1 | 5.28 ± 0.17 | 10.15 ± 0.56 | 11.96 ± 1.26 | 18.50 ± 1.44 |
| OVSAHO | 20.43 ± 4.71 | 10.80 ± 1.28 | 9.81 ± 1.07 | 19.7 ± 5.8 |

Without wishing to be bound by theory, the growth inhibitory activity observed with compounds 19-AP1 and 19-AP2 suggests that the active eneones are released because reduction of the exocyclic double bond in compounds with α-methylene-γ-butyrolactone resulted in a complete loss of activity;

### Example 11: CDK Inhibitor Assay

Secondary amine-based prodrugs generated from compound 19 engage RELA and IKKβ in cells and inhibit cancer cell growth. Without wishing to be bound by theory, it is hypothesized that prodrugs of compound 19 generated with secondary amine containing inhibitors that target a complimentary pathway will allow simultaneous targeting of two pathways. To test this hypothesis, two prodrugs each of compound 19 (compounds 5 and 7) and parthenolide (compounds 6 and 8) with CDK4/6 inhibitor palbociclib (compounds 5 and 6) were synthesized. Palbociclib has been approved for the treatment of metastatic breast cancer patients and AT7519 (compounds 7 and 8), which is a pan-CDK inhibitor currently in clinical trials.

To evaluate the change in vitro efficacy of the palbociclib hybrids (compounds 5 and 6) for CDK4 and CDK6, cell-free assays were performed (data summarized in Table 3, below). Although the CDK6:palbociclib co-crystal structure (pdb id: 5L21) suggests that the piperazine nitrogen atom is solvent exposed, palbociclib hybrids with compound 19 and parthenolide (compounds 5 and 6) were less active than palbociclib against CDK4 and CDK6. The compound 19-palbociclib hybrid (Compound 5) was ˜100- and ˜20-fold less potent than palbociclib against CDK4 and CDK6 respectively. The parthenolide-palbociclib hybrid (Compound 6) was ˜200- and ˜50-fold less potent than palbociclib against CDK4 and CDK6 respectively. It is clear that CDK6 tolerates alkylation of piperazine nitrogen atom better than CDK4.

TABLE 3

| | IC₅₀ (nM) | | |
|---|---|---|---|
| Kinase | Palbociclib | 5 | 6 |
| CDK4 | <0.38 | 46 ± 14 | 85 ± 18 |
| CDK6 | <0.38 | 9 ± 1 | 18 ± 1 |

Without wishing to be bound by theory, the larger size of parthenolide compared to compound 19 is probably responsible for a greater loss of activity of the parthenolide-hybrid (Compound 6) compared to compound 19-hybrid (Compound 5). Nevertheless, it is clear that blocking the piperazine nitrogen on palbociclib results in significant loss of activity, which will be regained in the cells upon the release of the NFκB inhibitors (Compound 19 and parthenolide).

### Example 12: Synergy Studies

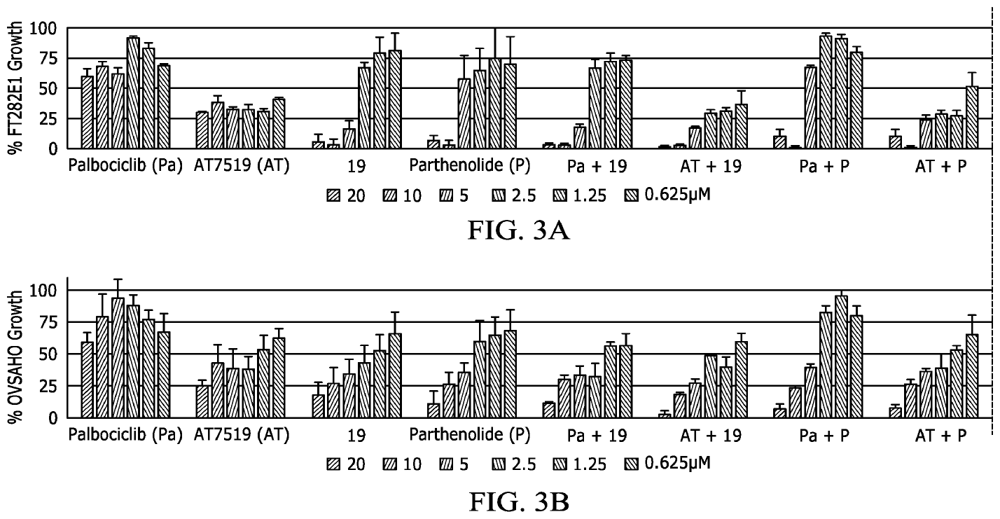

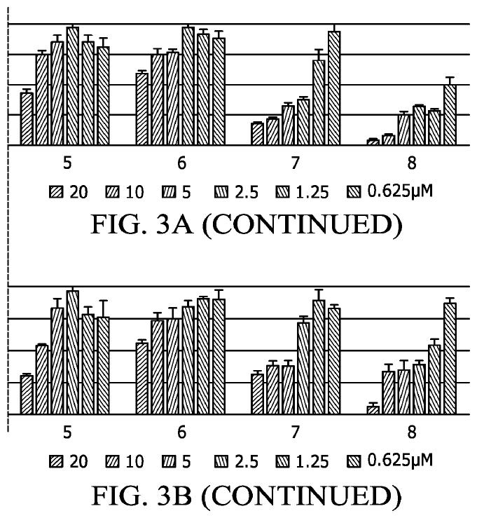

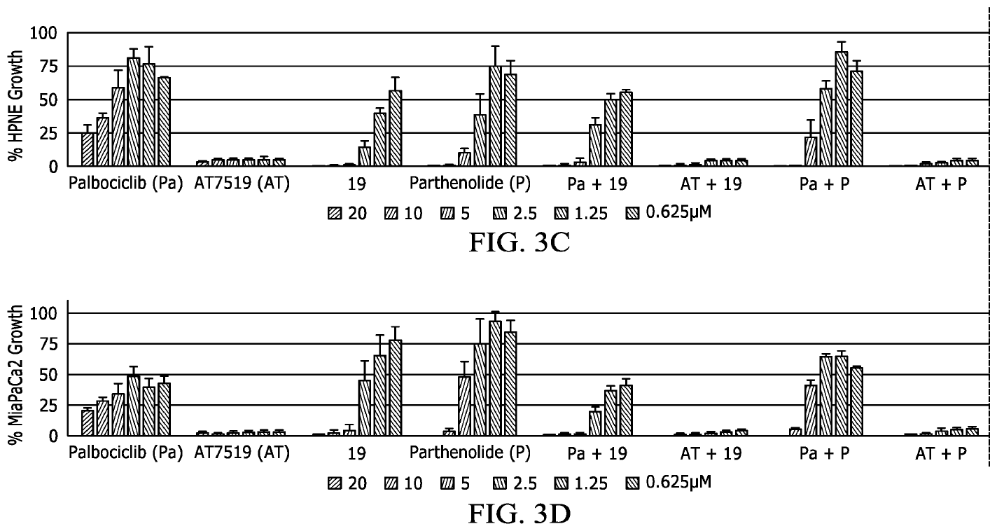

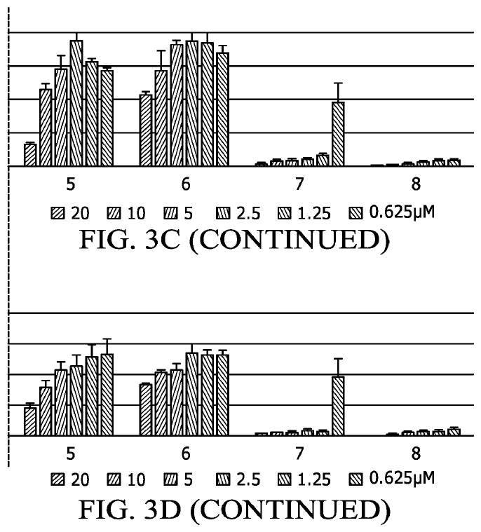

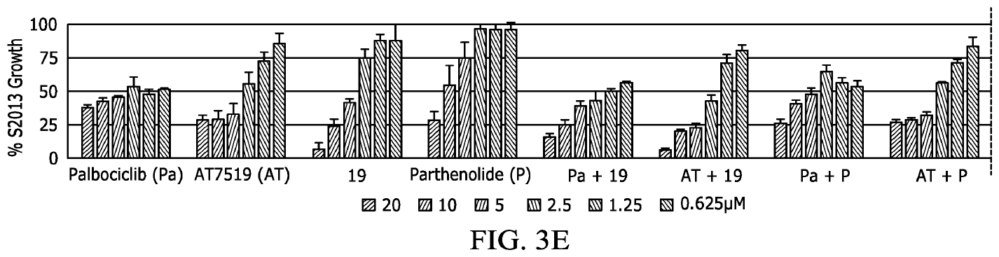

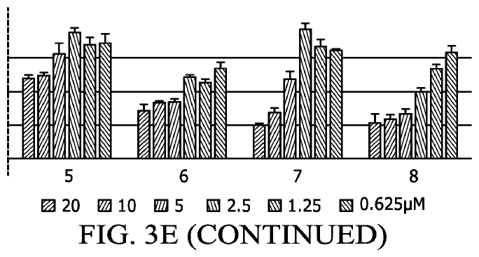

CDK4, CDK5, and CDK6 show a negative Pearson and Spearman correlation with RELA each with a p value<0.05 (depmap.org) suggesting that simultaneous targeting of these CDKs and RELA would result in synergistic effects. To test this idea, the effects of prodrugs comprising NFκB and CDK moieties (palbociclib, AT7519, compound 19, parthenolide) in the form of four compounds disclosed herein (compounds 5-8) and their corresponding 1:1 mixture in growth inhibition assays were studied in a panel of five cell lines (FIGS. 3A-3E).

Among the inhibitors treated as single agents the potency irrespective of the cell line ranked as follows: AT7519>compound 19>parthenolide>palbociclib. The 1:1 combination treatment showed dose-response across all cell lines, and the potency mirrored the dominant single agent treatment. Surprisingly, the compounds of the disclosure were less potent than the corresponding 1:1 combinational treatment. As expected, the AT7519 based compounds (compounds 7 and 8) were more potent than the palbociclib based compounds (compounds 5 and 6). Without wishing to be bound by theory, the potency trends of the hybrids followed those of the single treatments which suggests that the compounds are dissociating in the cells. The average combination index (^{Ave}CI) at effective dose (ED) 50, 75 and 90 for compound 5 in OVSAHO cells was 0.9 indicating weak synergism while the ^{Ave}CI>1.0 for the FT282E1 cells.

The efficacy of the palbociclib based compounds (compounds 5 and 6) with the corresponding (1:1) mixtures was studied in an expanded panel of 9 ovarian cancer (CaOV3, OVCAR5, OVCAR8, A2780, Kuramochi, OVCAR4, OVSAHO, SKOV3 and SNU119) and 2 immortalized fallopian tube epithelial cell lines (FT282E1 and FT282C11) (data summarized in Table 4, below).

TABLE 4

| | IC₅₀ (Mean ± SD □M) | | | |
|---|---|---|---|---|
| | Palbociclib + | | Palbociclib + | |
| Cell line | 19 | 5 | Parthenolide | 6 |
| FT282E1 | 9.8 ± 8.3 | 14.6 ± 1.2 | 9.1 ± 0.6 | >40 |
| FT282C11 | 17.6 ± 0.9 | 21.8 ± 1.5 | 9.8 ± 0.4 | 25.6 ± 4.8 |
| CaOV3 | 5.3 ± 0.5 | 5.5 ± 0.5 | 5.6 ± 0.2 | 25.0 ± 3.2 |
| OVCAR5 | 5.1 ± 0.5 | 9.3 ± 0.9 | 6.2 ± 0.2 | 32.6 ± 4.4 |
| OVCAR8 | 7.6 ± 0.7 | 9.1 ± 0.9 | 6.4 ± 0.5 | 38.4 ± 4.5 |
| A2780 | 9.6 ± 1.4 | 6.6 ± 1.3 | 6.1 ± 0.7 | 31.0 ± 7.0 |
| Kuramochi | 28.0 ± 3.0 | >40 | 9.8 ± 1.2 | >40 |
| OVCAR4 | 12.7 ± 0.7 | 17.9 ± 1.4 | 11.8 ± 2.4 | >40 |
| OVSAHO | 12.4 ± 4.0 | 11.4 ± 0.9 | 3.2 ± 0.2 | 31.1 ± 2.3 |
| SKOV3 | 14.2 ± 1.2 | 15.4 ± 1.5 | 4.5 ± 0.8 | >40 |
| SNU119 | 20.5 ± 2.1 | 8.8 ± 1.6 | 2.9 ± 0.4 | 34.9 ± 6.3 |

Analyses of the data show that on an average compound 6 was ˜7-fold less potent than the corresponding 1:1 mixture, while the compound 19-based compound 5 was equipotent to the corresponding 1:1 mixture. The ^{Ave}CI for compound 5 in SKOV3 cells was 0.2 indicating strong synergism.

Although the parthenolide:palbociclib (1:1) mixture was on an average ˜2-fold more potent than the compound 19:palbociclib (1:1) mixture, the compound 19-palbociclib compound 5 was ˜3-fold more potent than the parthenolide-palbociclib compound 6. Without wishing to be bound by theory, this could be attributed to lower potency of parthenolidepalbociclib compound 6 in the in vitro kinase assays; alternatively, the release of palbociclib from compound 19 may be more efficient than the release of palbociclib from parthenolide.

These results support the development of compounds with α-methylene-γ-butyrolactone moiety containing NFκB inhibitors and known drugs that have secondary amine functionality as a means to target two pathways simultaneously.

### CONCLUSION

In conclusion, two amine-based prodrugs of compound 19, which was previously reported as an NFκB pathway inhibitor, were synthesized. The prodrugs were more stable to biological nucleophiles, and the associated slow release was characterized by ¹⁹F NMR studies. Using an alkyne-tagged prodrug (Compound 4), engagement of NFκB pathway proteins was demonstrated for the first time by an analog with α-methylene-γ-butyrolactone moiety in cells. The prodrugs exhibited anticancer effects in growth inhibition assays.

Screening compounds disclosed herein in cell lines revealed that compound 19-derived compounds were more potent than parthenolide derived compounds both in cell free and cell-based assays. The compounds disclosed herein are valuable tools for advancing reactive compounds with α-methylene-γ-butyrolactone moiety as NFκB inhibitors.

In view of the many possible embodiments to which the principles of the disclosure may be applied, it should be recognized that the illustrated embodiments are only examples and should not be taken as limiting the scope of the invention.

What is claimed:

1. A compound, or pharmaceutically acceptable salt thereof, having the structure of Formula I: 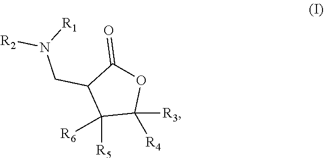
wherein
each of R₁ and R₂ is independently C₁₋₆ alkyl or C₁₋₆ alkylene-Y₁—X—Y₂—Z, or
R₁ and R₂ together with the nitrogen atom to which they are attached form a 5-7 membered heterocycloalkyl ring optionally having 1 additional ring heteroatom selected from O, S, and N, wherein the heterocycloalkyl ring is substituted with —CF₃ or —Y₁—X—Y₂—Z;
R₃ is H; or
R₃ and R₄ together with the carbon atom to which they are attached form a 5-7 membered heterocycloalkyl ring having 1-2 ring heteroatoms selected from O, S, and N, wherein the heterocycloalkyl ring is optionally fused to a C₅₋₁₀ aryl ring;
R₅ is H, or
R₅ and R₄ together with the carbon atoms to which they are attached form a C₆₋₁₂ cycloalkyl, wherein the cycloalkyl ring is optionally fused to a 3-5 membered heterocycloalkyl ring having 1-2 ring heteroatoms selected from O, S, and N, and the cycloalkyl ring is optionally substituted with 1-2 R₇;
wherein one of R₃ and R₅ is not H;
R₆ is H;
each R₇ is independently H or C₁₋₆ alkyl;
each of Y₁ and Y₂ is independently a bond, —NR₇—, or —C(O)NR₇—;
X is 5-6 membered heteroaryl having 1-2 ring heteroatoms selected from O, S, and N, or C₆₋₁₀ aryl;
Z is 6-10 membered heteroaryl having 1-3 ring heteroatoms selected from O, S, and N, 12-14 membered heterocycloalkyl having 1-3 ring heteroatoms selected from O, S, and N, or C₆₋₁₀ aryl, each substituted with 1-4 R₃; and
each R₃ is independently halo, OH, C₁₋₆ alkyl, C₅₋₆ cycloalkyl, C(O)NH₂, or C(O)CH₃.

2. The compound or salt of claim 1, wherein
each of R₁ and R₂ is independently C₁₋₆ alkyl, or
R₁ and R₂ together with the nitrogen atom to which they are attached form a 5-7 membered heterocycloalkyl ring optionally having 1 additional ring heteroatom selected from O, S, and N, wherein the heterocycloalkyl ring is substituted with —CF₃ or —Y₁—X—Y₂—Z;
R₃ is H; or
R₃ and R₄ together with the carbon atom to which they are attached form a 5-7 membered heterocycloalkyl ring having 1-2 ring heteroatoms selected from O, S, and N, wherein the heterocycloalkyl ring is optionally fused to a C₆₋₁₀ aryl ring;
R₅ is H, or
R₅ and R₄ together with the carbon atoms to which they are attached form a C₆₋₁₂ cycloalkyl, wherein the cycloalkyl ring is optionally fused to a 3-5 membered heterocycloalkyl ring having 1-2 ring heteroatoms selected from O, S, and N, and the cycloalkyl ring is optionally substituted with 1-2 R₇;
wherein one of R₃ and R₅ is not H;
R₆ is H;
each R₇ is independently H or C₁₋₆ alkyl;
each of Y₁ and Y₂ is independently a bond, —NR₇—, or —C(O)NR₇—;
X is 5-6 membered heteroaryl having 1-2 ring heteroatoms selected from O, S, and N;
Z is 6-10 membered heteroaryl having 1-3 ring heteroatoms selected from O, S, and N, or C₅₋₁₀ aryl, each substituted with 1-4 R₈; and
each R₃ is independently halo, OH, C₁₋₆ alkyl, C₅₋₆ cycloalkyl, or C(O)CH₃.

3. The compound or salt of claim 1 or 2, wherein R₁ and R₂ together with the nitrogen atom to which they are attached form a 6 membered heterocycloalkyl ring optionally having 1 additional ring N heteroatom.

4. The compound or salt of claim 3, wherein R₁ and R₂ together with the nitrogen atom to which they are attached form a 6 membered heterocycloalkyl ring having 1 additional ring N heteroatom.

5. The compound or salt of any one of claims 1 to 4, wherein the heterocycloalkyl ring is substituted with —Y₁—X—Y₂—Z.

6. The compound or salt of any one of claims 1 to 5, wherein R₃ and R₄ together with the carbon atom to which they are attached form a 5-7 membered heterocycloalkyl ring having 1-2 ring heteroatoms selected from O, S, and N, wherein the heterocycloalkyl ring is optionally fused to a C₆₋₁₀ aryl ring, and R₅ is H.

7. The compound or salt of claim 6, wherein R₃ and R₄ together with the carbon atom to which they are attached form a 5 membered heterocycloalkyl ring having 1 ring N heteroatom, wherein the heterocycloalkyl ring is fused to a phenyl ring.

8. The compound or salt of any one of claims 1 to 5, wherein R₅ and R₄ together with the nitrogen atom to which they are attached form a C₆₋₁₂ cycloalkyl, wherein the cycloalkyl ring is optionally fused to a 3-5 membered heterocycloalkyl ring having 1-2 ring heteroatoms selected from O, S, and N, and the cycloalkyl ring is optionally substituted with 1-2 R₇, and R₃ is H.

9. The compound or salt of claim 8, wherein R₅ and R₄ together with the carbon atoms to which they are attached form a C₁₀ cycloalkyl, wherein the cycloalkyl ring is optionally fused to a 3 membered heterocycloalkyl ring having 1 ring O heteroatom.

10. The compound or salt of claim 9, wherein the cycloalkyl ring is substituted with 1 or 2 methyl.

11. The compound of any one of claims 1 to 10, wherein at least one of Y₁ and Y₂ is —NR₇— or —C(O)NR₇—.

12. The compound or salt of any one of claims 1 to 11, wherein X is 5 membered heteroaryl having 2 ring N heteroatoms or 6 membered heteroaryl having 1 ring N heteroatom.

13. The compound or salt of any one of claims 2 to 12, wherein Z is 10 membered heteroaryl having 3 ring N heteroatoms, or phenyl.

14. The compound or salt of any one of claims 1 to 13, selected from the group consisting of compounds 5, 6, 7, 8, 9, and 10:

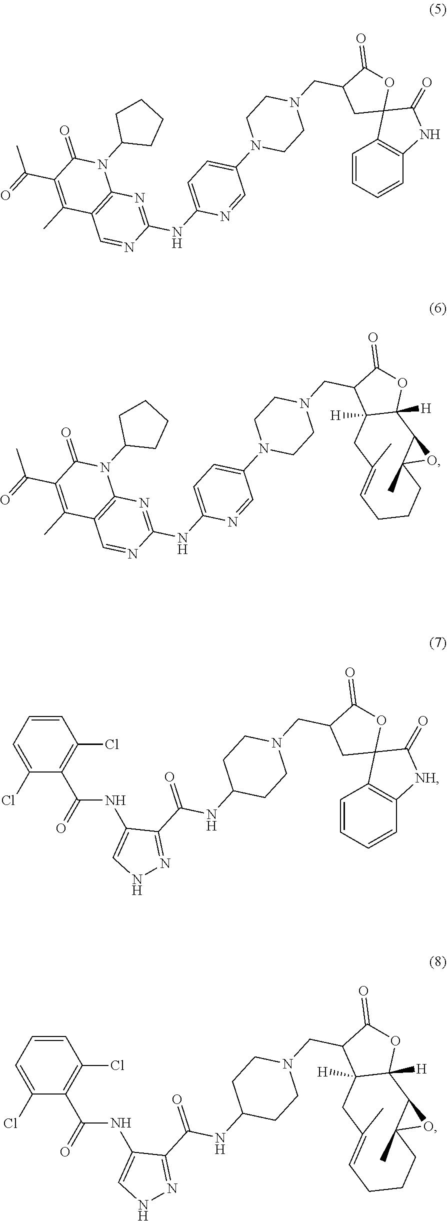 [SMILES: CC(=O)c1c(C)c2cnc(Nc3ccc(N4CCN(CC5CC6(OC5=O)C(=O)Nc5ccccc56)CC4)cn3)nc2n(C2CCCC2)c1=O ; CC(=O)c1c(C)c2cnc(Nc3ccc(N4CCN(CC5C(=O)O[C@@H]6[C@H]7O[C@]7(C)CCC/C=C(\C)C[C@@H]56)CC4)cn3)nc2n(C2CCCC2)c1=O ; O=C(NC1CCN(CC2CC3(OC2=O)C(=O)Nc2ccccc23)CC1)c1n[nH]cc1NC(=O)c1c(Cl)cccc1Cl ; C/C1=C\CCC[C@@]2(C)O[C@@H]2[C@H]2OC(=O)C(CN3CCC(NC(=O)c4n[nH]cc4NC(=O)c4c(Cl)cccc4Cl)CC3)[C@@H]2C1]

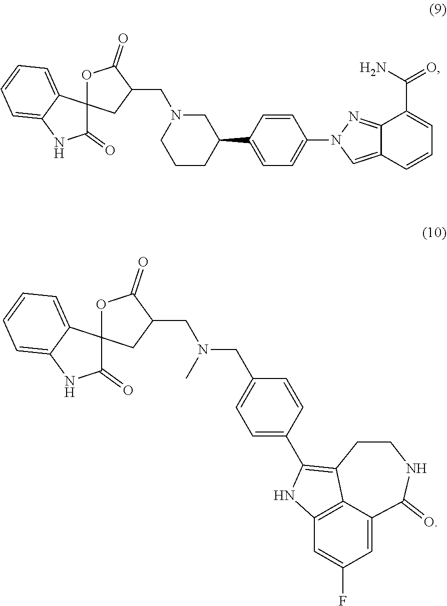 [SMILES: NC(=O)c1cccc2cn(-c3ccc([C@H]4CCCN(CC5CC6(OC5=O)C(=O)Nc5ccccc56)C4)cc3)nc12 ; CN(Cc1ccc(-c2[nH]c3cc(F)cc4c3c2CCNC4=O)cc1)CC1CC2(OC1=O)C(=O)Nc1ccccc12]

15. The compound or salt of claim 14, selected from the group consisting of compounds 5, 6, 7, and 8:

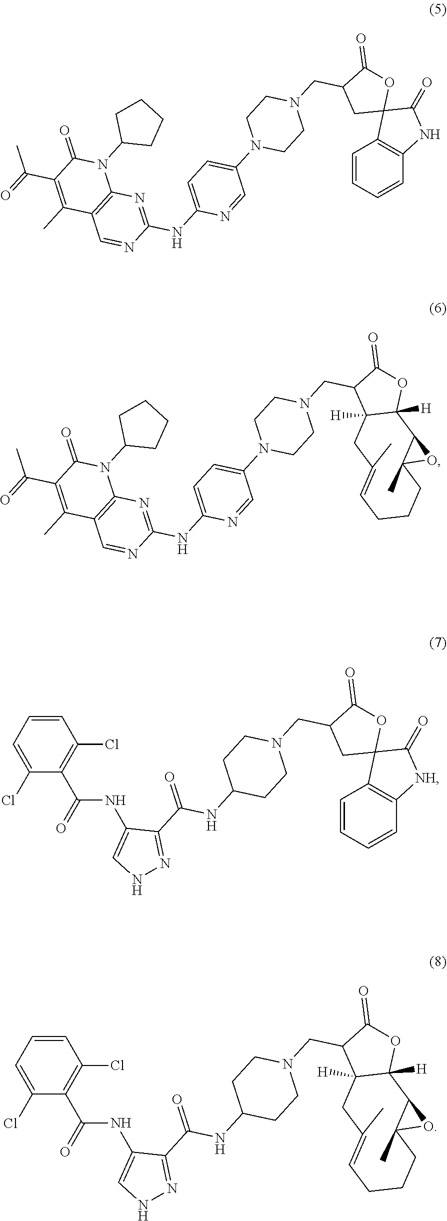 [SMILES: CC(=O)c1c(C)c2cnc(Nc3ccc(N4CCN(CC5CC6(OC5=O)C(=O)Nc5ccccc56)CC4)cn3)nc2n(C2CCCC2)c1=O ; CC(=O)c1c(C)c2cnc(Nc3ccc(N4CCN(CC5C(=O)O[C@@H]6[C@H]7O[C@]7(C)CCC/C=C(\C)C[C@@H]56)CC4)cn3)nc2n(C2CCCC2)c1=O ; O=C(NC1CCN(CC2CC3(OC2=O)C(=O)Nc2ccccc23)CC1)c1n[nH]cc1NC(=O)c1c(Cl)cccc1Cl ; C/C1=C\CCC[C@@]2(C)O[C@@H]2[C@H]2OC(=O)C(CN3CCC(NC(=O)c4n[nH]cc4NC(=O)c4c(Cl)cccc4Cl)CC3)[C@@H]2C1]

16. The compound or salt of claim 1, selected from the group consisting of compounds 19-AP1 and 19-AP2:

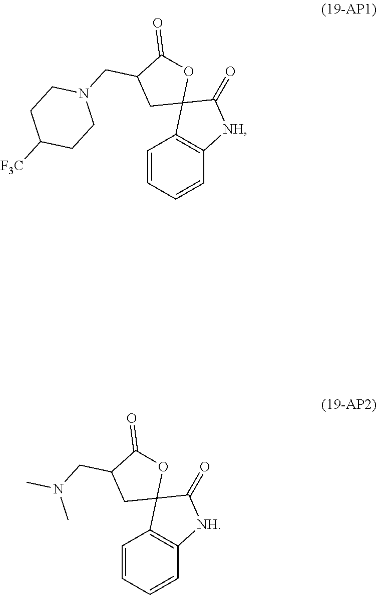 [SMILES: O=C1OC2(CC1CN1CCC(C(F)(F)F)CC1)C(=O)Nc1ccccc12 ; CN(C)CC1CC2(OC1=O)C(=O)Nc1ccccc12]

17. A pharmaceutical composition comprising the compound or salt of any one of claims 1 to 16 and a pharmaceutically acceptable carrier or excipient.

18. A method of treating or preventing a disease or disorder, comprising administering to a subject in need thereof a therapeutically effective amount of the compound or salt of any one of claims 1 to 16 or the pharmaceutical composition of claim 17.

19. The method of claim 18, wherein the disease or disorder is selected from the group consisting of cancer, autoimmune diseases, inflammatory diseases, diabetes, cardiovascular diseases, and neurological diseases.

20. The method of claim 19, wherein the disease or disorder is cancer.

21. The method of claim 19 or 20, wherein the disease or disorder is ovarian cancer.

22. The method of any one of claims 18 to 21, wherein the subject is human.
